(19) United States
(12) Patent Application Publication
(10) Pub. No.: US 2019/0022229 A1
(43) Pub. Date: Jan. 24, 2019

(54) CANNABINOID/TERPENE ENRICHED CAPRYLIC ACID

(71) Applicant: Annabelle Manalo, Franklin, TN (US)

(72) Inventor: Annabelle Manalo, Franklin, TN (US)

(21) Appl. No.: 16/111,975

(22) Filed: Aug. 24, 2018

Related U.S. Application Data
(63) Continuation-in-part of application No. 16/040,530, filed on Jul. 19, 2018.
(60) Provisional application No. 62/535,633, filed on Jul. 21, 2017.

(51) Int. Cl. A61K 47/12; A61K 31/05

(52) U.S. Cl. CPC A61K 47/12; A61K 31/05

(57) ABSTRACT

A chemical composition for human consumption includes a cannabinoid composition mixed in a delivery system, wherein the delivery system can be any of: oil based, water soluble, nano, edible, or capsule. The product has a total amount or per dose, ranging from 1 mg to 5000 mg of individually isolated material, wherein the individually isolated material can be any of cannabinoids and terpenes, such as CBD alone, THC alone, or Limonene alone, but is not limited thereto. The product can also include the use of the individually isolated material blended with other cannabinoids or with terpenes. The product can be blended with both cannabidiol and other cannabinoids and/or terpenes. This includes both the acid and decarboxylated forms of cannabinoids and terpenes. Also, this includes Caprylic Acid (C8) in a liquid or oil or powdered form embodying a purity of 99.9% or below.

### CROSS REFERENCE TO RELATED APPLICATIONS

This application claims the priority of Provisional Application No. 62/535,633 filed on Jul. 21, 2017, inventor Annabelle Manalo, entitled “CANNABIDIOL-ENRICHED CAPRYLIC ACID”. The entire disclosure of this provisional patent application is hereby incorporated by reference thereto, in its entirety; and claims the priority of Utility Patent application Ser. No. 16/040,530 filed on Jul. 19, 2018, inventor Annabelle Manalo, entitled “CANNABIDIOL-ENRICHED CAPRYLIC ACID”. The entire disclosure of this Utility patent application is hereby incorporated by reference thereto, in its entirety.

### STATEMENT REGARDING FEDERALLY SPONSORED RESEARCH OR DEVELOPMENT

Not applicable.

### FIELD OF THE INVENTION

The present invention pertains to the field of Cannabinoid and Terpene Enriched Delivery by Caprylic Acid. More particularly, the invention pertains to a more efficient and higher absorption rate of any and all delivery mechanisms of cannabis through the pairing of Caprylic Acid (C8).

### BACKGROUND OF THE INVENTION

The invention embodies the enhanced delivery of all cannabis (marijuana and hemp especially but not exclusive to) derived molecules. Cannabinoids and terpenes can be individually isolated for a molecule's maximum benefits or can be selectively blended in order to encourage an entourage effect. The association of any of the above with caprylic acid makes for an increased bioavailability of cannabinoids and terpenes into the human body.

The present disclosure relates generally to cannabinoid and terpene/terpenoid-enriched caprylic acid for human consumption. More particularly, but not exclusively, the present disclosure pertains to a composition comprising cannabidiol (CBD) in caprylic add (C8) and a method for mixing cannabidiol in caprylic acid.

Cannabinoids are chemical compounds that bind to chemical receptors in the human body. Cannabinoids bind to specific cellular receptors called cannabinoid receptors. This system is known as the endocannabinoid system and results in the binding of both endogenous and exogenous compounds to cellular cannabinoid receptors. Cannabinoid compounds can be classified into three groups: endocannabinoids, phytocannabinoids, and synthetic cannabinoids. Phytocannabinoids are chemical compounds concentrated in the oily resin of plants such as cannabis and hemp. Over 100 phytocannabinoids have been identified, many of which show beneficial medical properties.

Cannabinoids can be administered to the human body through multiple routes including inhalation, oral ingestion, transdermal absorption, and sublingual absorption. Currently, common practice includes isolating phytocannabinoid compounds and diluting the isolates in an oily medium that can be consumed by sublingual absorption. The isolated phytocannabinoid compounds can either be either specifically isolated to obtain a single phytocannabinoid compound (single isolate) or be isolated to obtain a plurality of heterogeneous phytocannabinoid compounds (whole-plant extract). Single isolated cannabinoids are advantageous due to the elimination of unwanted compounds such as tetrahydrocannabinol (THC), a psychoactive compound that under current federal law cannot be present at concentrations above 0.03%. Nonpsychoactive cannabinoids, such as cannabidiol, have no psychoactive side effects and avoid toxicity often encountered with the high doses of THC useful in treating certain medical diseases.

The use of oils as a delivery method commonly includes oils such as coconut oil or MCT oil. MCT oil is made up of medium-chain triglycerides, or MCTs, and generally includes triglycerides having chains of carbon molecules ranging from 6 to 12 carbons long. MCTs used for oil-based delivery systems are mostly extracted from coconuts and coconut oil. MCTs are converted to ketones and used as fuel in the body, however, they can commonly cause undesired side effects like digestive issues. Caprylic acid, an eight-carbon chain acid, is converted to ketones and is less susceptible to causing digestive issues. Caprylic acid is the rarest MCT but is also odorless and flavorless, making it ideal as a delivery system for human consumption.

Due to its low molecular weight, caprylic acid passes through the blood-brain barrier upon polymorphic transformation into beta-hydroxybutyrate (BHB) via interaction with an enzyme to modulate a ketone. Passage through the blood-brain barrier permits targeting to the central nervous system. Other MCTs such as capric acid (C10) must be cleaved at the methyl group before being converted into a ketone homologous to BHB, but this process must take place in the liver due to expression and location of necessary enzymes to carry out the reaction. Studies show a lower requirement for glucose to modulate this conversion, suggesting that the skeletal muscles benefit more from capric acid (C10) and the brain and CNS benefit more from caprylic acid (C8). This is further supported by caprylic acid having a suppressive effect on glycolysis and increasing neuronal metabolism. This activity has shown favorable results for treatment of cancers and seizures which use glucose as a nutrient source.

Ultra-refined caprylic acid provides a reliable energy source for the body through rapid absorption and conversion into ketones. Additionally, caprylic acid can pass through the blood-brain barrier more quickly than other MCTs, which can suppress excessive glycolysis. Due to its molecular structure, caprylic acid is not stored as body fat and is metabolized more efficiently than sugar. The graph shown in FIG. 1, taken from Vandenberghe, C. et al., Acute plasma ketone response to coconut oil alone or in combination with different medium chain triglycerides, ISSFAL Congress, Banf Canada (2016), demonstrates the differences between caprylic acid and other medium-chain triglycerides on the total plasma ketone levels over an eight-hour period. In this experiment, caprylic acid increased ketones by approximately three times more than capric acid (C10) alone.

Recent studies have demonstrated that ketone bodies provide the brain and central nervous system with energy and prevent the degradation of white matter in the brain to procure other energy sources. Caloric restriction studies have shown that during reduced caloric intake, a shift in energy sources, from glucose to ketones, occurs and supplies the brain with the necessary amount of energy. The reduced availability of glucose causes a switch to the use of ketones, which has shown preservation of white matter integrity as well as enhanced long-term memory due to caloric restriction.

Current commercial formulations of cannabinoid compounds mixed in oil-based delivery systems are limited in effectiveness due to a maximum concentration level based on the oil type used as the solvent, due to limited delivery of the cannabinoid into the body, and due to the side effects of both the cannabinoids and oil-based substrate.

### SUMMARY OF THE INVENTION

From the foregoing, it is seen that it is a problem in the art to provide a device meeting the above requirements. According to the present invention, a formulation is provided, and a method of making and delivering the formulation is provided, which meets the aforementioned requirements and needs in the prior art.

Specifically, the formulation and method according to the present invention provides a more diverse relationship as compared with that of the parent Utility patent application noted above. Here, the pairing of all cannabinoids, whether isolated or in an extract or in a distillate form, with caprylic acid provides the most efficient delivery system. Human and Cellular data on absorption rates, efficacy, and purity support this, as discussed further below.

The present invention pertains to the field of Cannabinoid and Terpene Enriched Delivery by Caprylic Acid. More particularly, the invention pertains to a more efficient and higher absorption rate of any and all delivery mechanisms of cannabis through the pairing of Caprylic Acid (C8).

Applicant identified a need for a CBD oil product containing a high concentration of CBD in an efficient delivery system. Applicant discovered that cannabinoid formulations within certain oil-based delivery systems, when properly paired, can overcome these limitations with increased concentrations, increased absorption rates, and decreased side effects. One aspect of the invention uses caprylic acid as that delivery system. Applicant found that use of single isolate caprylic acid as a CBD delivery system is highly effective and surprisingly delivers physiological results superior to currently available products. Applicant surprisingly found that use of caprylic acid as a CBD delivery system permits greater concentrations of CBD per volume, decreases delivery time to the body and central nervous system, decreases side effects, and decreases or even eliminates the need for flavoring agents.

From the foregoing, it is seen that it is a problem in the art to provide a compound, formulation, and method meeting the above requirements. According to the present invention, a compound or formulation and method is provided which meets the aforementioned requirements and needs in the prior art. Specifically, the present disclosure relates generally to cannabinoid and terpene/terpenoid-enriched caprylic acid for human consumption. More particularly, but not exclusively, the present disclosure pertains to a composition comprising cannabidiol (CBD) in caprylic acid (C8) and a method for mixing cannabidiol in caprylic acid.

The present disclosure relates, in one embodiment, to a chemical composition for human consumption. The chemical composition may include a cannabinoid composition mixed in an oil-based delivery system. The cannabinoid composition may include cannabidiol (CBD) and the oil-based delivery system may include caprylic acid. In a preferred embodiment, the chemical composition comprises CBD oil and caprylic acid.

The present disclosure also relates, in another embodiment, to a method of producing a chemical composition for human consumption. In a preferred embodiment, the method includes the steps for mixing a cannabinoid composition into an oil-based delivery system. A further aspect of the preferred embodiment comprises a method for mixing 3000 milligrams (mg) of cannabinoid per one (1) ounce volume of the oil-based delivery system. In certain preferred embodiments, the method comprises the steps for mixing 3000 mg of cannabidiol per one (1) ounce caprylic acid.

This includes any product of a total or per dose amount ranging from 1 mg to 5000 mg of individually isolated cannabinoids or terpenes, and this also includes any blend of such cannabinoids and terpenes. The present invention also includes any blend of such cannabinoids and terpenes together.

There are hundreds of cannabinoids, examples being CBD and THC, and the present invention is not limited to only those two examples. This invention is contemplated to encompass and include all of the single isolated and defined molecules that are cannabinoids and can come from a cannabis plant.

Also, there are hundreds of terpenes, one example being Limonene, and the present invention is not limited to only this example. This invention is contemplated to encompass and include all of the single isolated and defined molecules that are terpenes and can come from a cannabis plant.

For purposes of discussion herein, where CBD, THC, and Limonene are used, it will be understood that they are exemplary, and that other named molecules that are cannabinoids as well as other named molecules that are terpenes can be used.

Further, this invention includes the use of the above-noted individually isolated material that is blended with both cannabinoids and terpenes. In this case, the cannabinoids and terpenes can be in any relative proportions.

Other objects and advantages of the present invention will be more readily apparent from the following detailed description when read in conjunction with the accompanying drawings.

### BRIEF DESCRIPTION OF THE DRAWINGS

FIG. 1 is a graph whose source is identified above, which demonstrates the differences between caprylic acid and other medium-chain triglycerides on the total plasma ketone levels over an eight-hour period.

### DETAILED DESCRIPTION OF THE INVENTION

The present invention pertains to the field of Cannabinoid and Terpene Enriched Delivery by Caprylic Acid. More particularly, the invention pertains to a more efficient and higher absorption rate of any and all delivery mechanisms of cannabis through the pairing of Caprylic Acid (C8).

By the present invention and as set forth in the above-noted parent Utility patent application, cannabinoids have an enriched delivery in pairing with caprylic acid.

This pairing can be delivered through: a water-soluble manner such as vape, smoke, dab, and so on; in oil form; in a powder form; in edibles; in capsules; and can be applied nasally and rectally.

Single isolated cannabinoids are advantageous due to the elimination of all other compounds. In the case of CBD, for example, the ability to remove psychoactive compounds is beneficial. Under current federal law, a CBD product is considered marijuana once THC concentrations above 0.03% are present. Terpenes or terpenoids are a diverse set of organic building blocks produced from within the same glands of the cannabis flower in which cannabinoids arise. Terpenes are considered the aromatic constituent of the plant. There are over 100 known terpenes that are believed to play a synergistic role with cannabinoids, specifically THC and CBD.

Tetrahydrocannabinol (THC) is the principal psychoactive component of a Cannabis plant. THC promotes the release of dopamine within the brain causing a euphoric effect. Research shows positive impact on cognitive function and a variety of diseases. There are over 113 other known cannabinoids but only a few have been defined.

The present invention contemplates the following as being within the scope of the present invention: Each individual terpene, cannabinoid, or full cannabis (hemp or marijuana) extract paired with caprylic acid.

This includes any product of a total amount or per dose ranging from 1 mg to 5000 mg of individually isolated cannabinoids or terpenes (examples of which including, but not limited to, CBD alone, THC alone, or Limonene alone); and/or blended cannabinoids or terpenes.

This includes both the acid and decarboxylated forms of cannabinoids and terpenes. Specifically, this includes cannabinoid acids and cannabinoids, as well as, terpenes and terpenoids. Also, this includes Caprylic Acid (C8) in a liquid or oil or powdered form embodying a purity of 99.9% or below.

This includes any product of a total or per dose amount ranging from 1 mg to 5000 mg of individually isolated cannabinoids or terpenes, and this also includes any blend of such cannabinoids and terpenes. The present invention also includes any blend of such cannabinoids and terpenes together.

There are hundreds of cannabinoids, examples being CBD and THC, and the present invention is not limited to only those two examples. This invention is contemplated to encompass and include all of the single isolated and defined molecules that are cannabinoids and can come from a cannabis plant.

Also, there are hundreds of terpenes, one example being Limonene, and the present invention is not limited to only this example. This invention is contemplated to encompass and include all of the single isolated and defined molecules that are terpenes and can come from a cannabis plant.

For purposes of discussion herein, where CBD, THC, and Limonene are used, it will be understood that they are exemplary, and that other named molecules that are cannabinoids as well as other named molecules that are terpenes can be used.

Further, this invention includes the use of the above-noted individually isolated material that is blended with both cannabinoids and terpenes. In this case, the cannabinoids and terpenes can be in any relative proportions.

The formulation of the present invention has the ability to bypass the first metabolic step in which interaction with metabolic enzyme can be avoided, ensuring no side effects that may arise with supplements or pharmaceutical medications that need metabolic attention within the liver.

List of Major Cannabinoids (this Listing is not Exhaustive nor Limited to those Listed Below):

Cannabidiol (CBD)

Cannabidivarin (CBDV)

Cannabichromevarin (CBCV)

Delta-9-tetrahydocannabinol (THC)

Tetrahydrocannabivarin (THCV)

Cannabigerol (CBG)

Cannabichromene (CBC)

Cannabigreivarin (CBGV)

Cannabicyclol (CBL)

List of Major Terpenes (this Listing is not Exhaustive nor Limited to those Listed Below):

| | |
|---|---|
| Linalool | Pinene |
| Limonene | Terpinene |
| Myrcene | Camphene |
| Terpineol | Nerolidol |
| Borneol | Ocimene |
| Caryophyllene | Sabinene |
| Eucalyptol | Pulegone |

Hypothesis

The dosing of the individual and/or combined cannabinoids and/or terpenes can be minimal based on the increased absorption of pairing with Caprylic Acid (C8). The pairing would enable the most efficient delivery of the above cannabinoids and terpenes, to name a few, into the human body with any delivery method of choice.

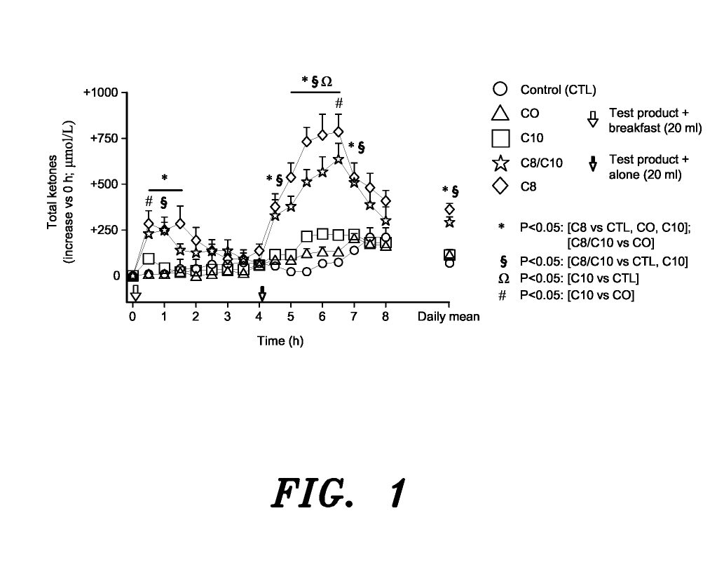

FIG. 1 is a graph whose source is identified above, which demonstrates the differences between caprylic acid and other medium-chain triglycerides on the total plasma ketone levels over an eight-hour period.

As used herein, “cannabinoid” includes any compound that interacts with a cannabinoid receptor and various cannabinoid mimetics, including naturally occurring compounds derived from plant extracts, and non-natural compounds which are semi-synthetically or synthetically produced. The cannabinoid may be included in its natural form, in the form of a pro-drug, or in the form of a pharmaceutically acceptable salt thereof. Additionally, the term “cannabinoid” is meant to include its natural form that has been isolated, purified, or modified, and synthetically derived cannabinoids that have been isolated, purified, or modified.

As used herein, “cannabidiol” refers to cannabidiol, cannabidiol prodrugs, pharmaceutically acceptable derivatives of cannabidiol, including pharmaceutically acceptable salts of cannabidiol, cannabidiol prodrugs, and cannabidiol derivatives.

In one embodiment the cannabinoid is substantially free from impurities. As used herein, “substantially free of impurities” shall mean that impurities, including any cannabinoid not intended to be administered in a therapeutically effective quantity, are present in an amount by weight of the composition of less than about 10%, less than about 5%, less than about 1%, less than about 0.1%, or less than about 0.01%.

In a further embodiment, the cannabidiol is substantially free from other cannabinoids. As used herein, “substantially free from other cannabinoids” shall mean that other cannabinoids, including any cannabinoid not intended to be administered in a therapeutically effective quantity, are present in an amount by weight of the composition of less than about 1%, less than about 0.1%, or less than about 0.01%.

In a further embodiment, the cannabinoids are extracted from a “full-hemp extract” and include a plurality of cannabinoids together in a single extract. A full-hemp extract may comprise a high concentration of CBD, greater than 50% weight/weight. Another embodiment may comprise CBD greater than 75% weight/weight, greater than 80% weight/weight, greater than 90% weight/weight, or greater than 95% weight/weight. A preferred embodiment comprises CBD greater than 99% weight/weight.

In a further embodiment, the cannabinoids are extracted from a “full-marijuana extract” and include a plurality of cannabinoids together in a single extract. A full-marijuana extract may comprise a high concentration of CBD, greater than 50% weight/weight. Another embodiment may comprise CBD greater than 75% weight/weight, greater than 80% weight/weight, greater than 90% weight/weight, or greater than 95% weight/weight. A preferred embodiment comprises CBD greater than 99% weight/weight.

In a further embodiment, the cannabinoids are extracted from a “full-marijuana extract” or a full-hemp extract, and include a plurality of cannabinoids together in a single extract. A full-marijuana extract may comprise a high concentration of THC and other cannabinoids, greater than 50% weight/weight. Another embodiment may comprise THC and other cannabinoids greater than 75% weight/weight, greater than 80% weight/weight, greater than 90% weight/weight, or greater than 95% weight/weight. A preferred embodiment comprises THC and other cannabinoids greater than 99% weight/weight.

As used herein, the term “oil” refers to a substance composed primarily of triglycerides of fatty acids. Oils may include vegetable oils extracted from various parts of the plant, including the seeds, fruit, or leaves of plants. In some embodiments the oils are derived from canola, rapeseed, palm, palm kernel, coconut, tucum, sunflower, safflower, olive, macadamia, babassu, castor, peanut, cotton, flaxseed, linseed, cohune, and jatropha. In further embodiments, the oils may be derived from a genetically modified plant.

“Medium chain fatty acids” as used herein refers to fatty acids containing 6 to 14 carbons, preferably 8 carbons. In certain preferred embodiments, the oil-based delivery system comprises an 8 carbon medium chain fatty acid, caprylic acid. In certain embodiments, the caprylic acid is substantially free from impurities. As used herein, “substantially free of impurities” shall mean that impurities, including any other medium chain fatty acid not intended to be administered in a therapeutically effective quantity, are present in an amount by weight of the composition of less than about 10%, less than about 5%, less than about 1%, less than about 0.1%, or less than about 0.01%, In some embodiments, impurities may comprise other medium chain fatty acids.

One embodiment described herein includes a chemical composition comprising a mixture of oil and a cannabinoid composition. In another embodiment, the oil comprises medium-chain triglycerides.

In another embodiment, the chemical composition comprises a mixture of oil, wherein the oil is a triglyceride having an eight-carbon chain backbone known as caprylic acid, and a cannabinoid composition. In yet another embodiment, the caprylic acid is substantially free from impurities such that the caprylic acid has less than about 10%, less than about 5%, less than about 1%, less than about 0.1%, or less than about 0.01% by weight of impurities. In certain embodiments, the impurities may comprise other medium-chain triglycerides.

In certain preferred embodiments, the chemical composition comprises cannabidiol. In another embodiment, the chemical composition comprises a full-hemp extract comprising cannabidiol. In yet another embodiment, the cannabidiol is isolated or purified from a plant source. In still another embodiment, the cannabidiol is substantially free from impurities and thus, the impurities are present in an amount by weight of the composition of less than about 1%, less than about 0.1%, or less than about 0.01%, In certain embodiments, the impurities may comprise other cannabinoids.

In certain preferred embodiments, the chemical composition comprises oil and cannabidiol wherein the cannabidiol is present at a concentration of at least 3000 mg per one (1) ounce volume of oil. In other embodiments, the chemical composition comprises caprylic acid and cannabidiol wherein the cannabidiol is present a concentration of at least 3000 mg per one (1) ounce volume of caprylic acid. In another embodiment, the chemical composition comprises oil and cannabidiol wherein the cannabidiol is present at a concentration of at least 2500 mg per one (1) ounce volume to 3000 mg per one (1) ounce volume of oil. In other embodiments, the cannabidiol is present at a concentration between 2500 mg per one (1) ounce volume to 3000 mg per one (1) ounce volume of caprylic acid.

In another embodiment, the chemical composition comprises oil and cannabidiol wherein the cannabidiol is present at a concentration of at least 1000 mg per one (1) ounce volume of caprylic acid. In another embodiment, the chemical composition comprises caprylic acid and cannabidiol wherein the cannabidiol is present at a concentration of at least 1000 mg per one (1) ounce volume of oil. In another embodiment, the chemical composition comprises oil and a cannabinoid compound present at a concentration of at least 1000 mg per one (1) ounce volume of oil. In a one embodiment, the method for making the chemical composition comprises providing an oil-based substrate, heating the oil-based substrate to a specified temperature, and mixing a cannabinoid compound into the oil-based substrate. In yet another embodiment, the specified temperature is maintained for a specific time interval after initial mixing. In yet another embodiment, mixing of the heated oil-based substrate and cannabinoid compound mixture continues during the specific time interval at which the mixture is maintained at the specified temperature.

In another embodiment, the oil-based substrate comprises medium-chain triglycerides. In yet another embodiment, the oil-based substrate comprises caprylic acid. In another embodiment, the oil-based substrate comprises caprylic acid that is substantially free from impurities wherein the caprylic acid has less than about 10%, less than about 5%, less than about 1%, less than about 0.1%, or less than about 0.01% by weight of impurities. In certain embodiments, the impurities may comprise other medium-chain triglycerides.

In other embodiments, heating the oil-based substrate comprises applying a heat source to the oil-based substrate so that the substrate reaches at least 30 degrees Celsius. In other embodiments, heating the oil-based substrate comprises heating to a temperature of at least 40 degrees Celsius. In another embodiment, heating the oil-based substrate comprises heating to a temperature to at least 50 degrees Celsius

In additional embodiments, the cannabinoid compound mixed with the oil-based substrate comprises a plurality of cannabinoid constituents. In other preferred embodiments, the cannabinoid compound comprises a single cannabinoid constituent. In yet another preferred embodiment, the cannabinoid compound comprises cannabidiol. In yet another embodiment, the cannabinoid compound comprises cannabidiol that is substantially free from impurities and thus, the impurities are present in an amount by weight of the composition of less than about 1%, less than about 0.1%, or less than about 0.01%. In certain embodiments, the impurities may comprise other cannabinoids. In another embodiment, the cannabinoid compound is a full-hemp extract.

In other embodiments, the mixture of the cannabinoid compound in the oil substrate is maintained at the specified temperature for at least fifteen minutes, at least twenty minutes, or at least twenty-five minutes. In certain embodiments, the mixture of the cannabinoid compound in the oil substrate is maintained at the specified temperature for between seventeen and eighteen minutes. In one aspect, the mixture of the cannabinoid compound in the oil substrate is maintained at the specified temperature for about 17.68 minutes. In certain embodiments, the mixture of the cannabinoid compound in the oil substrate is maintained at the specified temperature for between twenty-one and twenty-two minutes. In one aspect, the mixture of the cannabinoid compound in the oil substrate is maintained at the specified temperature for about 21.40 minutes. In certain embodiments, the mixture of the cannabinoid compound in the oil substrate is maintained at the specified temperature for between twenty-five and twenty-six minutes. In one aspect, the mixture of the cannabinoid compound in the oil substrate is maintained at the specified temperature for about 25.22 minutes.

In another embodiment, the method comprises heating MCT oil (55% C8, 36% C10, 0.2% Lauric Acid, 9% other fatty acids, total 14 g) to at least 50 degrees Celsius, mixing 500 mg of 99% CBD Isolate into 1.5 ounces (or 14.78 rnL) MCT oil (55% C8, 36% C10, 0.2% Lauric Acid, 9% other fatty acids, total 14 g,) and maintaining the heat and mixture for at least 21.40 minutes. In another embodiment, the method comprises heating caprylic acid (99% C8, total 14g) to at least 50 degrees Celsius, mixing 500 mg of 99% CBD Isolate into 1.5 ounces (or 14.78 mL) caprylic acid (99% C8, total 14g) and maintaining the heat and mixture for at least 21.40 minutes.

In preferred further embodiment, the method comprises heating MCT oil (55% C8, 36% C10, 0.2% Lauric Acid, 9% other fatty acids, total 14 g) to at least 50 degrees Celsius, mixing 3000 mg of 99% CBD Isolate into 1 ounce (or 29.56 mL) MCT oil (55% C8, 36% C10, 0.2% Lauric Acid. 9% other fatty acids, total 14 g), and maintaining the heat and mixture for at least 25.22 minutes. In another embodiment, the method comprises heating caprylic acid (99% C8, total 14a) to at least 50 degrees Celsius, mixing 3000 mg of 99% CBD Isolate into 1 ounce (or 29.56 mL) caprylic acid (99% C8. total 14g), and maintaining the heat and mixture for at least 17.68 minutes.

Sublingual administration of certain preferred embodiments of the invention yields faster absorption rates compared to current commercially available products. In one example, sublingual administration of a mixture of caprylic acid and cannabidiol with a concentration of approximately 3000 mg of cannabidiol per 1 ounce of caprylic acid demonstrated an approximately three-fold increase over time in cannabidiol levels in the blood as measured in ng/ml.

The invention being thus described, it will be evident that the same may be varied in many ways by anyone having skill in the applicable arts. Such variations are not to be regarded as a departure from the spirit and scope of the invention and all such modifications are intended to be included within the scope of the claims.

What is claimed is:

1. A chemical composition comprising:
a delivery system, wherein said delivery system comprises caprylic acid, or pharmaceutically acceptable salts thereof;
an individually isolated material selected from the group of cannabinoids and terpenes, mixed in the oil-based delivery system.

2. A chemical composition as claimed in claim 1, wherein said cannabinoids include THC and CBD in said individually isolated material.

3. chemical composition as claimed in claim 1, wherein said terpenes include Limonene blended with said individually isolated material.

4. A chemical composition as claimed in claim 1, wherein said delivery system includes any of a water-soluble manner such as vape, smoke, and dab; in oil form; in a powder form; in edibles; in capsules; and can be applied nasally and rectally.

5. A chemical composition as claimed in claim 4, wherein said cannabinoids and terpenes are in any relative proportions ranging from 0.001% to 0.999%.

6. A chemical composition as claimed in claim 1, further comprising any of cannabinoid acids and cannabinoids.

7. A chemical composition as claimed in claim 1, further comprising any of terpenes and terpenoids.

8. A chemical composition as claimed in claim 1, further comprising any of cannabinoid acids and cannabinoids, and any of terpenes and terpenoids.

9. A method for mixing cannabinoids, or pharmaceutically acceptable salts thereof, into an oil-based delivery system comprising the steps of:
a) providing an oil-based substrate comprising caprylic acid;
b) heating the oil-based substrate to a specified temperature;
c) mixing a cannabinoid compound into the heated oil-based substrate,
where said cannabinoid compound includes any of cannabinoid acids and cannabinoids, and any of terpenes and terpenoids; and
d) maintaining the specified temperature for a time-interval.

10. A method for sublingual administration of a mixture of caprylic acid and a specified material composed of any of cannabinoid acids and cannabinoids, and any of terpenes and terpenoids, in which said specified material has a concentration of approximately 3000 mg of cannabidiol per 1 ounce of caprylic acid, whereby there is an approximately three-fold increase over time in levels of said specified material in the blood as measured in ng/ml.
